(19) United States
(12) Patent Application Publication
(10) Pub. No.: US 2004/0076966 A1
(43) Pub. Date: Apr. 22, 2004

(54) Method and system for the co-isolation of cognate DNA, RNA and protein sequences and method for screening co-isolates for defined activities

(76) Inventors: J. Brian Windsor, Austin, TX (US); Alan M. Lloyd, Austin, TX (US)

Correspondence Address: Kevin A. Gust, Thompson & Knight, L.L.P., Suite 1200, 98 San Jacinto Blvd., Austin, TX, 78701, US

(21) Appl. No.: 10/284,072

(22) Filed: Oct. 30, 2002

Related U.S. Application Data
(60) Provisional application No. 60/340,105, filed on Oct. 30, 2001.

(51) Int. Cl. C12Q 1/68; C07H 21/04; C12N 1/08

(52) U.S. Cl. 435/6; 435/270; 536/25.4

(57) ABSTRACT

The present invention provides a method and a system for the co-isolation of cognate DNA, RNA and protein sequences, a method for screening co-isolates for defined activities, and the production of operons for simultaneous or coordinated expression of genes. Encapsulating solutions and microstructures in lipid vesicles or polymer capsules permits transcription/translation reactions amenable to techniques and instruments that utilize an aqueous environment. Microstructures containing binding sites for both mRNA and protein and encapsulated into a liposome or hollow polymer capsule, creating a reactosome. After transcription/translation, or other reactions, the reactosomes may be screened using aqueous methods. Once the desired reactosomes are isolated, the vesicles are disrupted, and the microspheres can be collected. Once transcription/translation reactions occur, the capsules may be frozen for storage or later use. Additionally, co-isolates may be screened for a desired activity before transcription/translation occurs. Operons may be produced in the capsules, separated, and isolated for later use.

### BACKGROUND OF THE INVENTION

1. Field of the Invention

The present invention relates generally to the field of identifying proteins and genes (as well as other cellular molecules, such as metabolites), the co-isolation of DNA or RNA sequences and the proteins encoded by such sequences, and the production of operons for a simultaneous or coordinated expression of genes.

2. Description of Related Art

Several techniques have been developed for the cloning of genes based on identification of the protein expressed by the gene of interest. The most common technique is to transform a suitable bacteria such as E. coli with a gene library in a phage or plasmid vector, plate the bacteria on media selective for transformants, and screen plaques or colonies for the protein of interest. Common methods for identifying proteins include antibody binding, functional complementation of host proteins, and reaction with compounds in the media.

Generally, once the protein is identified in a colony or plaque, the expressed gene of interest is isolated from the host. In some cases, host cells are screened for expression of a gene of interest in single cell format using techniques such as Fluorescence Activated Cell Sorting (FACS) or by viewing on glass slides.

The expression of genes in a heterologous host is used not only to isolate naturally occurring genes from organisms, but also to identify products of recombinant DNA techniques such as gene shuffling and mutagenic PCR. Disadvantages of heterologous gene expression include the expression is limited to those genes which the host is able to express, and the technique is laborious and time consuming, involving bacterial cultures and the use of multiple agar plates. In U.S. Pat. No. 6,242,211, Peterson et al discloses a variation of the typical heterologous screening technique, wherein recombinant expression constructs, derived from pools of diverse species of organisms, are expressed in a host which is suspended with other host cells in a semi-solid matrix (macrodroplets) containing a reporter for identification of metabolic products. As Peterson requires a host, it would be desirable to develop a method that does not need a host.

Phage display (Smith, G. P. 1985, Science 228:1315-1317) permits isolation of a peptide of interest that is expressed on the surface of a bacteriophage. However, this approach is limited to assays for protein binding and is usually limited to libraries of small peptides derived from short oligonucleotide sequences.

Other techniques utilize in vitro transcription/translation mixes that contain all the components necessary for transcription of DNA into mRNA and subsequent translation into protein. Some in vitro transcription/translation mixes and fractions of pools of cDNA are used to identify proteins of interest and, through multiple rounds of fractionation, the cDNA that encodes the protein. For example, the PROfusion technique (Kreider, B. L. 2000, Med Res Rev 20(3): 212-215) utilizes a peptide linker to bind a protein translated via in vitro transcription/translation to its mRNA, thus linking peptide and coding sequence.

Techniques have been developed for the compartmentalization of in vitro transcription/translation mixtures using water in oil emulsions. The Genescis technique (Tawfik, D. S. and Griffiths, A. D. 1998. Nature Biotechnology 16: 652-656) and the STABLE technique (Doi, N. and Yanagawa, H. 1999. FEBS Lett. 457(2): 227-230) both permit in vitro transcription/translation to occur in the aqueous compartments of water-in-oil emulsions. The STABLE technique couples the nascent peptide within a compartment to its gene via a streptavidin-biotin linkage. The Genescis technique proposes coupling the substrate for a desired reaction product to the genes within the compartments and assaying de-emulsified transcription/translation products for the converted target product and responsible gene. A particular disadvantage with these techniques is the water-in-oil emulsions, which are incompatible with existing flow cytometry, FACS instruments, and other systems that are water-based. It is therefore desirable to use lipid vesicles or polymer capsules that are amenable to techniques and instruments that utilize an aqueous environment.

Related to the topic of gene expression is the study of operons as coordinators for gene activity in prokaryotes.

Operons are regions of DNA in prokaryotes where multiple genes are under the control of a single promoter allowing for the simultaneous or coordinated expression of those genes. Operons may be used in bacteria for the expression of multiple genes in metabolic engineering techniques where it is advantageous or necessary to investigate the activity of multiple gene products within a single host. As operons are not present in the eukaryotic nuclear genome, multiple genes in eukaryotes cannot be investigated using the same techniques as for prokaryotes. It is therefore desirable to provide a technique for manufacturing and coordinating operons in eukaryotic organisms.

### SUMMARY OF THE INVENTION

The present invention overcomes the shortcomings of prior art techniques for identifying and isolating genes and proteins.

In one broad respect, the present invention is directed to a method for isolating DNA, RNA, and protein sequences, the method comprising the steps of formulating a solution comprising one or more components for in vitro transcription/translation reactions; and encapsulating said solution such that transcription/translation reactions proceed inside said capsule. In a narrow respect, the capsule is a lipid vesicle, and the lipid vesicle may be adapted for identification based on a fluorescent activity. In another narrow respect, the capsule is a polymer capsule, which may be adapted for identification based on a fluorescent activity. In another narrow respect, the step of encapsulating said solution such that transcription/translation reactions proceed inside said capsule further may comprise encapsulating one or more microspheres inside said capsule, wherein said one or more microspheres are adapted to bond with one or more protein sequences. In a narrower respect, the step of encapsulating said solution such that transcription/translation reactions proceed inside said capsule further may comprise encapsulating one or more microspheres inside said capsule, wherein said one or more microspheres are adapted to bond with one or more gene sequences. In other narrower respects, the gene sequences may comprise RNA, cDNA, or DNA. In another narrow respect, the method may further comprise the step of isolating one or more transcription/translation reaction products from disrupted said one or more capsules. In some narrow respects, the components are obtained from a eukaryote source or a prokaryote source.

In another broad respect, the present invention is directed to a method for screening co-isolates, said method comprising the steps of formulating a solution comprising one or more components; encapsulating the solution, said capsule containing the solution hereinafter referred to as a reactosome; screening said reactosome for one or more specific activities; and isolating said one or more reactosomes. In some narrow respects, the solution comprises a reporter for a metabolic pathway or a substrate for a metabolic pathway. In some narrow respects, the capsule comprises a lipid vesicle or a polymer capsule. In another narrow respect, the step of isolating said one or more reactosomes comprises isolating said one or is more reactosomes with metabolite product of enzymatic reactions. In another narrow respect, the method further comprises the step of isolating one or more genes responsible for metabolite production. In a narrower respect, the step of screening said reactosome for one or more specific activities comprises screening said reactosome for fluorescent activity. In another narrower respect, the step of screening said reactosome for one or more specific activities comprises screening said reactosome for color. In another narrower respect, formulating said solution comprises adding one or more microspheres.

In another broad respect, the present invention is directed to a method for selecting microspheres exhibiting a desired activity, the method comprising the steps of: formulating a solution comprising one or more components, said one or more components comprising bound microspheres, screening said bound microspheres; and separating bound microspheres exhibiting one or more specific activities. In a narrow respect, the bound microspheres are manufactured by the method of formulating a solution comprising one or more components for in vitro transcription/translation reactions; and encapsulating the solution in a polymer capsule such that transcription/translation reactions proceed inside said polymer capsule. In another narrow respect, the bound microspheres are bound with a small molecule. In another narrow respect, screening said bound microspheres comprises screening for fluorescent activity. In another narrow respect, separating said bound microspheres comprises selecting said bound microspheres based on fluorescent activity.

In another broad respect, the present invention is directed to a method for generating one or more gene sequences adapted for use as an operon, said method comprising the steps of: obtaining a promoter-linked cDNA of selected size; phosphorylating one or more ends of said promoter-linked cDNA; ligating said promoter-linked cDNA such that a cDNA concatamers is formed; and extending a selected primer complementary to said promoter-linked cDNA. In one narrow respect, the method further comprises generating double-stranded cDNA.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a flow chart of the process of creating a protein/mRNA-bound microsphere library from an organism, according to one embodiment of the present invention.

FIG. 2 is an illustration of a reactosome according to one embodiment of the present invention.

FIG. 3 is an illustration of a liposome made from two different fluorescently dyed lipids in an approximately equal ratio according to one embodiment of the present invention.

FIG. 4 is a drawing of a microsphere containing generic protein binding site and polyT binding site for mRNA according to one embodiment of the present invention.

FIG. 5 is a drawing of a microsphere containing generic protein binding site and sequence-specific binding site for DNA according to one embodiment of the present invention.

FIG. 6 is a drawing of a microsphere containing His-tagged protein binding site and polyT binding site for mRNA according to one embodiment of the present invention.

FIG. 7 is a drawing of a microsphere with alternative binding site for protein and mRNA according to one embodiment of the present invention.

FIG. 8 is a flow chart of a possible application for microspheres bound using the present invention.

FIG. 9 is a flow chart of one possible application for bound microspheres encapsulated into a reactosome, according to one embodiment of the present invention.

### DETAILED DESCRIPTION OF EMBODIMENTS

The present invention overcomes the shortcomings of the prior art with methods of identifying proteins and genes (as well as other cellular molecules, such as metabolites) and the co-isolation of DNA or RNA sequences and the proteins encoded by such sequences. The identification of proteins and genes generally entails encapsulating a gene library in lipid vesicles or polymer capsules together with components for in vitro transcription/translation so that each vesicle/capsule contains one gene or desired combination of genes, introducing in each vesicle/capsule a microsphere or microspheres containing binding sites for protein and mRNA, and directing the transcription and translation of genes within the encapsulated areas.

### (i) Generation of Protein/Corresponding mRNA-Bound Microspheres

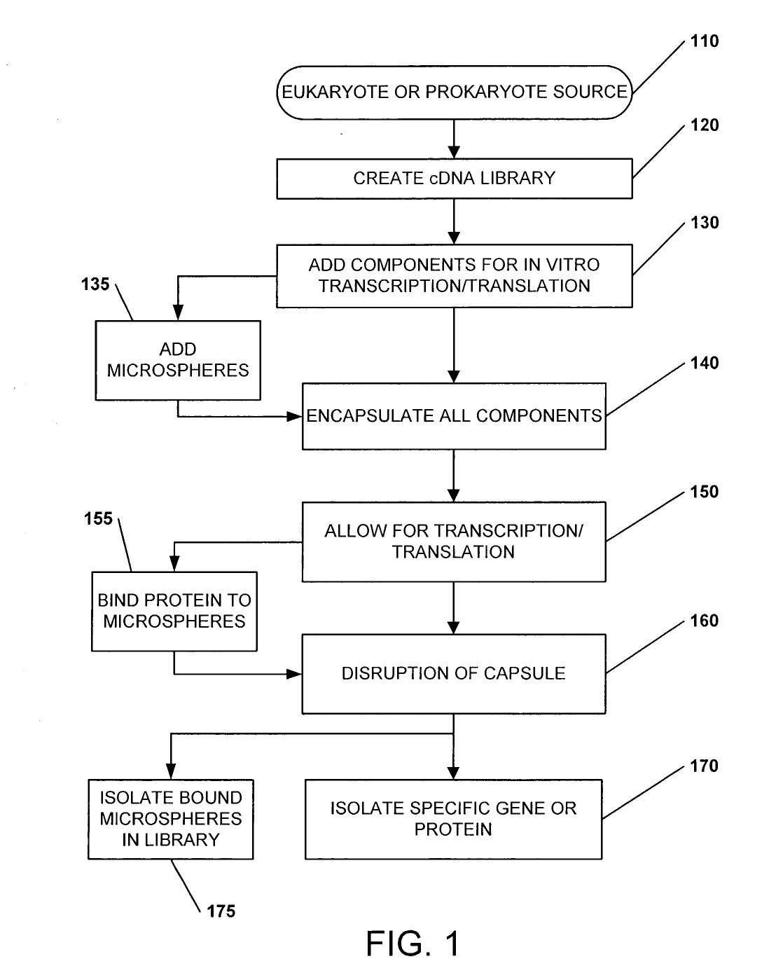

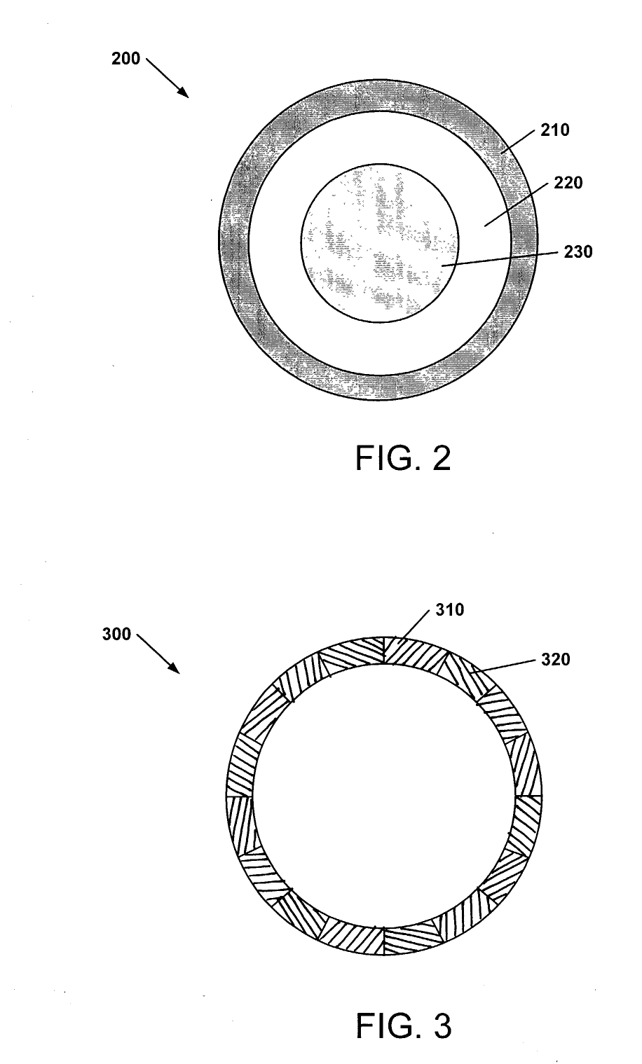

Referring to the figures, FIG. 1 is a flow chart 100 of a process of generating a protein/corresponding mRNA-bound microsphere library from an organism, according to one embodiment of the present invention. Using this process, protein/mRNA-bound microspheres are generated to facilitate the isolation of a specific gene or protein. The microspheres are capable of binding with mRNA and protein from all the genes of a library of genes, from an organism or synthetic preparation. Each microsphere binds with the mRNA and protein of a single gene or group of genes from the library. This process, depicted as a flow chart in FIG. 1, generally involves encapsulating the gene library in lipid vesicles, as shown in FIG. 2, (liposomes) 210 or polymer capsules together with components for in vitro transcription/translation so that each vesicle/capsule 200 contains one gene or desired combination of genes. In some embodiments, one or more microspheres may be included in a capsule, so that the resulting capsule is referred to as a reactosome. Any size vesicle/capsule 200 may be used, although the number of genes included in a liposome 210 or vesicle/capsule 200 would ideally be determined by calculating the amount of DNA present and the average volume of a liposome 210 or vesicle/capsule 200. In a preferred embodiment, microspheres 230 would optimally be of a diameter or volume that is some percentage of the inner diameter or volume of the liposome 210 or vesicle/capsule 200 and that allows for adequate transcription/translation reactions. In one embodiment a compartment 220 size of a 1-2 micron diameter is sufficient for this procedure.

Large unilamellar vesicle (LUV) liposomes may be prepared by a variety of methods, including but not limited to extrusion, sonication, reverse-phase evaporation, and swelling from a lipid-coated roughened Teflon® disc.

In preferred embodiments, liposomes 210 would be composed of lipids that have a temperature transition below the optimal temperature for in vitro transcription/translation. There are many lipid combinations which could serve these purposes, including, but not limited to, the following lipid combination: 70% 1,2-Dimyristoyl-sn-glycero-3-phosphocholine and 30% 1,2-Dimyristoyl-sn-glycero-3-[phosph-rac-(1-glycerol)] (sodium salt).

In some embodiments, fluorescent-tagged lipids facilitate identification using FACS or other such instruments. Unique “liposome fluorescence profiles” may be created by adjusting the concentration of a single fluorescent lipid, or by adjusting the ratio of two or more different fluorescent lipids. For example, as shown in FIG. 3, profiles of NBD 320 or lissamine rhodamine 310-tagged lipids can be created. NBD 320 has an excitation of 460 nm and an emission of 534 nm, while lissamine rhodamine 310 has an excitation of 550 nm and an emission of 590 nm. Using 100% of the NBD label would result in a specific fluorescence profile 310. Using 100% of the lissamine rhodamine label would yield a different profile 310. Using 50% NBD and 50% lissamine rhodamine would yield yet a third profile 330.

The in vitro transcription/translation components may be obtained from a variety of third party suppliers in kit form, and the specific components of a selected kit would be based on the parameters of the experiment. In one embodiment, the in vitro transcription/translation kit for genes expressed from a viral T7 promoter is Promega's TNT® T7 Quick Coupled Transcription/Translation System. However, it will be obvious to those skilled in the art that other translation/transcription kits, including those with several different promoters, may be used without departing in scope from the present invention.

In an alternative embodiment, hollow polymer capsules may be prepared according to the method of Caruso et al without departing in scope from the present invention.

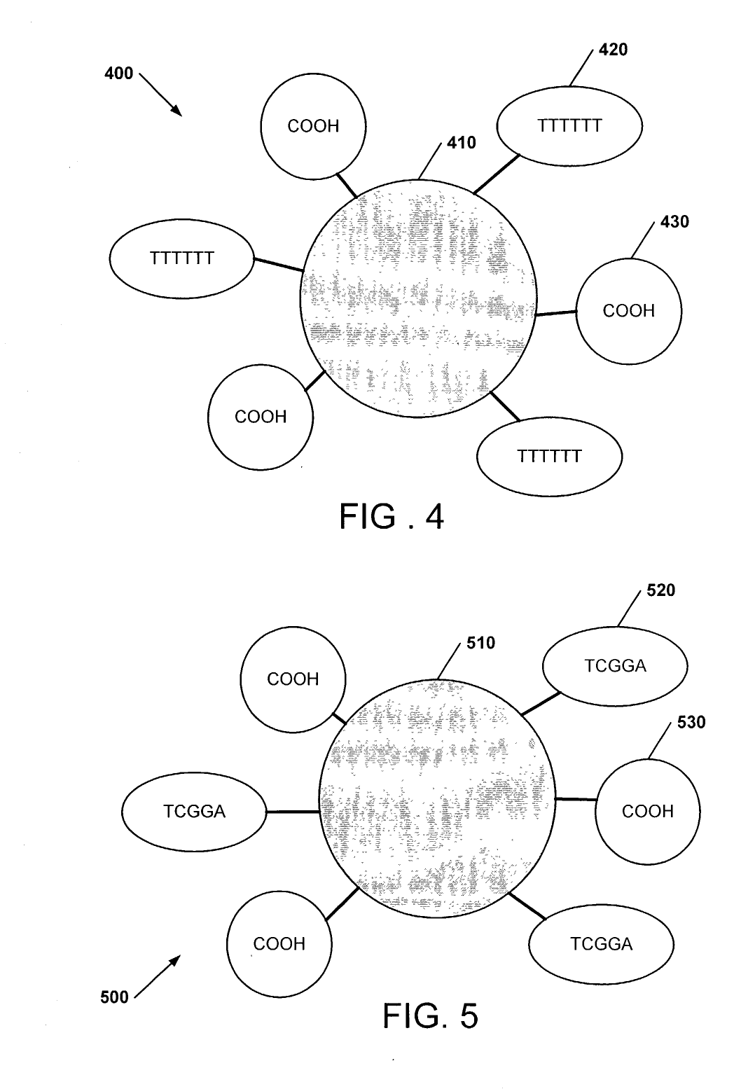

In step 130, a microsphere 230 (such as polystyrene beads) is incorporated in a solution containing DNA and in vitro transcription/translation mix. The microspheres 230 would optimally be of a diameter or volume that permits adequate transcription/translation reactions. In preferred embodiments, beads of 0.3-0.5 micron diameters may provide a sufficient compartment 220 for transcription/translation to occur. Microspheres 230 may be modified to possess surface chemistries, as shown in FIGS. 4-7, which permit for binding of mRNA (or alternatively DNA) and protein. Examples of surface modified microspheres are shown in FIGS. 4-7. In each example, the microsphere 410, 510, 610, and 710 ideally possesses at least two different binding groups—one for nucleotide 420, 520, 620, and 720, and one for protein 430, 530, 630, and 730.

As shown in FIG. 4, when binding mRNA, a binding group which ends with an oligo dT 420 nucleotide sequence may be used, as this would bind the polyA tail of mRNA sequences.

As shown in FIG. 5, DNA may be bound in a sequence specific fashion by modifying the microsphere 510 to possess a nucleotide group 530 that is complementary to the DNA sequence to be isolated. For oligo dT-primed cDNA, this sequence may be polyA. A general protein-binding group, such as a carboxylate, aldehyde, amine, tosyl, or other group, may be used for protein binding. However the choice of surface chemistry would be dependent on the specific experimental design and on the types of proteins desired for binding.

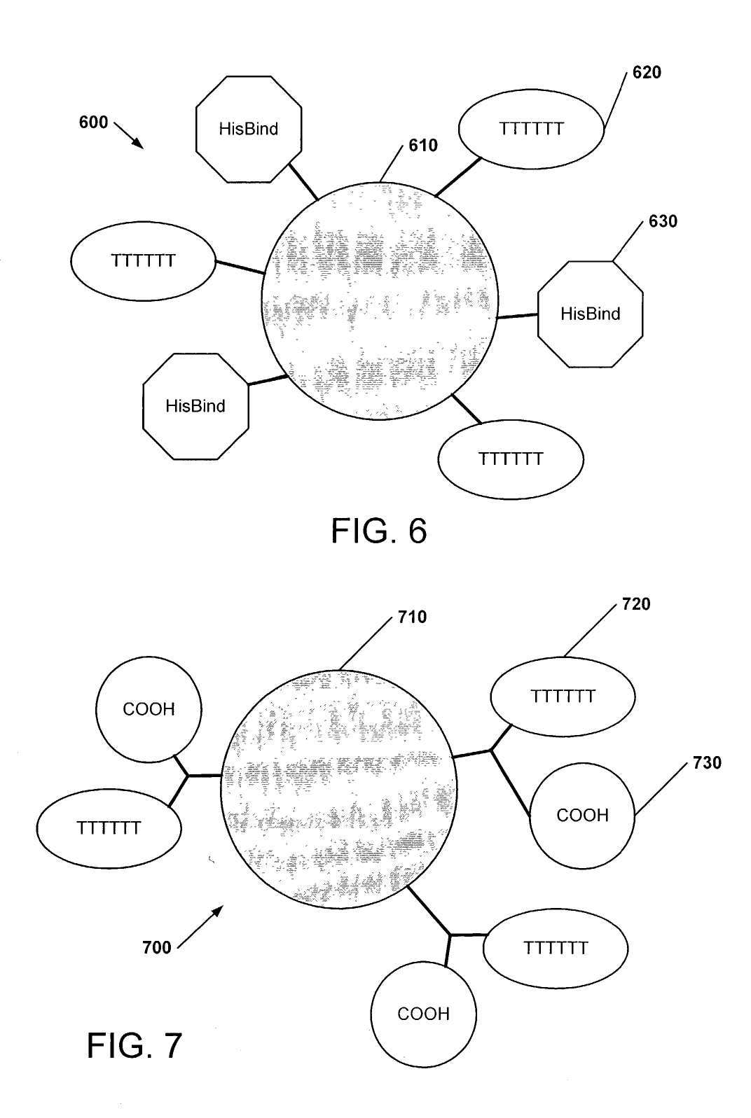

As shown in FIG. 6, in some embodiments, it may be desirable to use a His-tagged expressed protein and appropriate binding site 630 on a microsphere 610 for selective binding.

As shown in FIG. 7, in some embodiments, it may be desirable to use a microsphere 710 with a protein binding site 720, an mRNA (or alternatively DNA) binding site 730, and alternative binding sites 740 with further permit binding with mRNA (or alternatively DNA) or protein.

The binding of proteins and mRNAs to microspheres as described is useful in generating libraries of proteins and their coding sequences from cDNA or similar libraries. Such libraries may contain beads with a single mRNA/protein per bead or multiple random or selected mRNA/protein combinations. The nucleotide/protein combinations may occur naturally or be generated through protein evolution techniques such as DNA shuffling or mutagenic PCR. Microsphere libraries may contain gene sequences only for use in gene profiling techniques, or proteins for protein profiling techniques. An advantage to the present invention is that no knowledge of an organism's genome or proteome is needed in order to generate a library of gene-protein linked microspheres. Another advantage is that a stable record of all the expressed genes and proteins from an organism may be obtained even when samples of the organism are hard to obtain. Such libraries would be particularly useful in the discovery of metabolic enzymes from rare or exotic plant, marine, or other species.

Still referring to FIGS. 4-7, the microspheres 230 would be added to a desired solution at a concentration such that each vesicle/capsule 200 or liposome 210 would receive a single or defined number of microspheres. Those of skill in the art will appreciate that this concentration is calculated similarly to the calculation for DNA concentration.

As shown in FIG. 1, step 140, a reaction solution containing the microspheres 230, in vitro transcription/translation mix, and selected DNA, would be encapsulated into liposomes 210 or vesicles/capsules 200. In a preferred embodiment, liposomes that possess a net negative charge are used because they would not attract DNA or would not interfere with the transcription/translation reaction.

In step 150, the transcription/translation reaction would be allowed to proceed for a length of time sufficient for transcription/translation and binding of the protein to the microsphere 230.

In step 160, once the transcription/translation reaction and protein binding to the microspheres 230 is complete, the liposomes 210 or vesicles/capsules are disrupted and the bound microspheres are collected using one of various techniques. For example, if the microspheres 230 used were magnetic, the microspheres 230 could be collected using a magnet. Microspheres 230 may also be collected using centrifugation. It will be obvious to those skilled in the art that any technique for collecting the microspheres 230 may be used without departing in scope from the present invention.

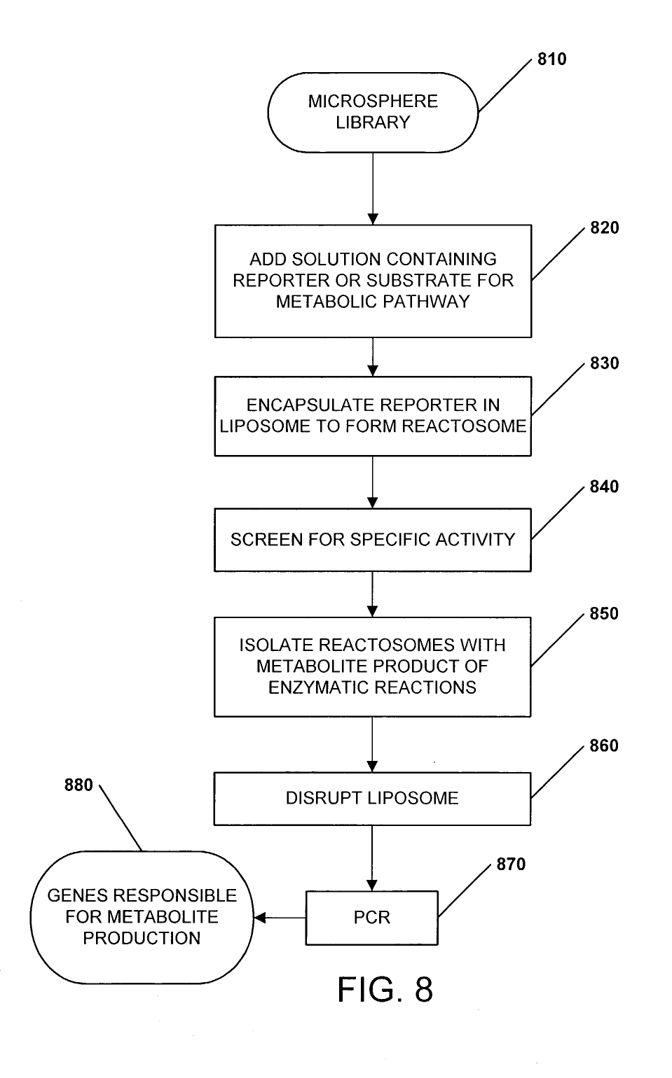

FIG. 8 is a flowchart of a method for isolating genes responsible for metabolite production, according to one embodiment of the present invention, in which bound microspheres are encapsulated into reactosomes, screened for a specific activity, isolated, and the liposomes are then disrupted to release genes responsible for metabolite production.

In FIG. 8, step 820, a solution containing a reporter or substrate for a desired metabolic pathway is prepared. This solution may possess properties (such as a specific pH or temperature) or contain molecules (such as antibodies, fluorescently-tagged molecules, nucleotides, synthetic or natural chemicals, or other molecules) which will allow for detection of a specific protein or proteins (or subunits) and/or nucleotide(s) (including catalytic nucleotides) based upon physical properties of the protein(s) and/or nucleotide(s) such as size, the ability to bind to a molecule; catalytic or enzymatic abilities such as the ability to cleave, react with, or otherwise interact with or alter a peptide, chemical, or other molecule; or other parameters characteristic of the specific protein(s) and/or nucleotide(s).

In step 830, the reporter is encapsulated in a liposome 210 to form a reactosome.

In step 840, the reactosome is screened for a specific activity, using a FACS or any other screening system. Detection of a specific protein or nucleotide, or the catalytic or enzymatic activity thereof, may be accomplished using any techniques that measure properties such as fluorescence, luminescence, color, UV absorption, mass, binding, migration on a gel, paper, capillary tube, etc. In some embodiments, the protein(s) and/or nucleotide(s) possess properties in and of themselves, such as fluorescence (e.g. GFP or a similar protein that is fluorescent), which would facilitate detecting their presence without special conditions or the addition of special molecules to the solution. In other embodiments, a fluorescently-tagged antibody which is specific to a peptide may be added to facilitate detection.

In some embodiments, a chemical substrate may be added which, when acted upon by an enzyme or catalytic nucleotide, allows for detection based on one or more methods described above. One such example is the cleavage of fluorescein di-β-D-galactopyranoside by the β-galactosidase enzyme. In this case, fluorescein di-β-D-galactopyranoside is a substrate molecule for the β-galactosidase enzyme and is cleaved by the enzyme. The substrate itself is not fluorescent. However, once cleaved the resulting product is fluorescent and may be readily detected. Adding fluorescein di-β-D-galactopyranoside (or similar substrate) to the solution would be requisite for the detection of a β-galactosidase enzyme via detection of the fluorescent product.

In other embodiments, a chemical, having a specific property such as a specific fluorescence wavelength emission, may be added, which would be converted through the action of an enzyme or catalytic nucleotide to a chemical molecule having a property which is different from the substrate and which is detectable using one or more of the methods described. This may be particularly useful in dissecting steps of metabolic pathways where a metabolite is the product of several enzymatic actions. An example would be the conversion of dihydroquercetin to quercetin via flavanol synthase. Both dihydroquercetin and quercetin are fluorescent when exposed to UV light, however the emission spectra of the 2 molecules are different. The flavanol synthase enzyme may be obtained by adding dihydroquercetin to the solution and look for quercetin based on its emissions.

It is also possible to assay for the quenching of a fluorescent substrate. In other embodiments, a drug which has a particular fluorescence or other such detectable property may be added, and the resulting solution may be observed for binding of the drug to a protein or nucleotide.

In step 850, the reactosomes with metabolite products of enzymatic reactions are isolated.

In step 860, the liposome 210 is ruptured using any technique known in the art.

In step 870 the proteins are screened using Polymerase Chain Reaction to identify the genes responsible for metabolite production 880.

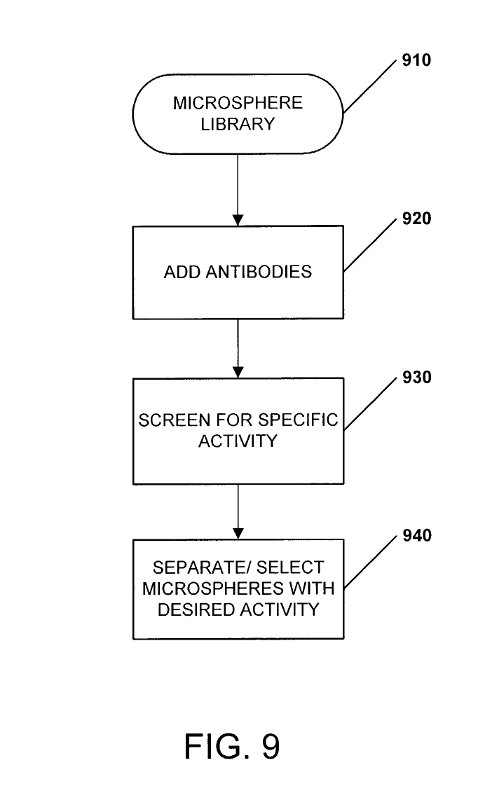

FIG. 9 is an illustration of one embodiment of the present invention, in which antibodies are used to isolate specific proteins or genes. Antibodies are added to a microsphere library. The microspheres are then screened for specific activity through a cell sorter. Once the microspheres have been sorted, the antibodies are then separated from the microspheres.

In step 920, antibodies are added to a microsphere library 810.

In step 930, the reactosome is screened for a specific activity, using a FACS or any other screening system.

In step 940, microspheres exhibiting a desired activity are separated and selected.

### (ii) Liposome or Polymer Capsule based In Vitro Transcription/Translation for use with Aqueous Phase Fluid Transport or Detection Devices

Liposomes 210 and polymer capsules containing gene products for use in assaying for particular activities with aqueous phase fluid transport or detection devices may be generated by modifying the above-described technique to exclude the use of microspheres in the reaction solution. Using this process, liposomes or polymer capsules sequester single genes or groups of genes in in vitro transcription/translation reactions. Completion of the in vitro transcription/translation reaction would yield gene products compartmentalized into vesicles or capsules. Unlike chambers of water-in-oil emulsions, the liposomes 210 or vesicles/capsules could subsequently be plated onto a glass slide, tethered to a solid surface, or used in FACS or other aqueous fluid flow devices. A FACS system such as Cytomation's MoFlo® High Throughput Sorter with CyCLONE® Automated Cloner is capable of depositing liposomes or vesicles/capsules into a 96-well format or sorting individual liposomes 210 or vesicles/capsules in a pattern onto glass slides. Sorted liposomes 210 may then be layered onto membranes adapted to capture protein and nucleotides (in the same array as the liposomes) and capable of capturing (or leaving) reaction products such as metabolites or other chemical compounds. The chemical compounds produced in individual liposome-encapsulated reactions may be identified using screening technology such as nanotech instrumentation utilizing gas chromatography, mass spec, high performance liquid chromatography.

The present invention possesses many advantages over prior technologies for the isolation of a particular protein or desired activity. Instead of being limited to the detection of specific activities, the present invention is capable of using reporters or substrate to product conversions can be detected that are completely soluble, as the liposome or capsule contains the reaction products. Since the liposomes or vesicles/capsules are easily manipulated, they may be used for identifying various activities.

### (iii) Generation of “Reactosome” consisting of a Bound Microsphere Encapsulated in a Liposome or Polymer Capsule for use in Identifying a Desired Activity that is Assayable by Means of a Soluble Reporter, Detector or Some other Non-Binding Event

Using some embodiments of the present invention, a protein activity or some other activity may be screened using an alternative form of “reactosome,” a unit consisting of a protein or nucleic acid bound microsphere, which may be produced by the procedure described in section (i), in a solution surrounded by a liposome or polymer capsule. The solution may contain one or more substrates for a desired activity produced by one or more enzyme reactions, or a reporter molecule for an activity. Although this method is similar to the method described in section (ii), there is no transcription/translation reaction step. In some embodiments, microspheres already bound with protein or nucleic acid are used instead of a transcription/translation step for the production of gene products. A desired activity could be assayed utilizing the instruments and techniques such as those described in section (ii), and this process would have the same advantages as those described in section (ii). The use of reactosomes as a means of assaying for and isolating a desired activity may be desirable in the dissection of metabolic pathways or the generation of novel metabolic pathways.

In some embodiments, microspheres bound with multiple proteins and the substrate of a known or desired multi-step enzymatic reaction may be used to isolate the microsphere containing the responsible gene sequences and protein products, after assaying for the reactosome possessing the end product of the reaction. Using this process, groups of proteins within a pathway or groups of proteins that produced desired or novel compounds may be identified. Such a system is adapted for use with soluble end products since the reactosome isolates the reaction and resulting end products.

### (iv) Generation of Promoter-Linked cDNA

In some embodiments of the present invention, promoter-linked cDNA is generated for use with in vitro transcription/translation. Since in vitro transcription/translation kits typically utilize specific promoter sequences, such as T7, having a means of obtaining promoter-specific cDNA may be desirable. In some embodiments, mRNA is obtained from an organism or sample to generate cDNA. In one embodiment, oligo dT-primed cDNA is generated from the mRNA using an oligo dT that is dephosphorylated at the 5′ end. The next step would involve removing the RNA using RNAse. A synthetic single stranded T7 promoter sequence is ligated from its 5′ phosphate to the 3′ end of the cDNA using T4 RNA ligase. The result of this ligation would be a pool of single-stranded cDNAs with T7 promoters and free T7 promoter sequences. Size selection would then be used to exclude unligated T7 promoters. Using a complementary T7 primer, the second strand of DNA could be generated to make the cDNA double stranded. The resulting product could be cloned into a vector.

### (v) Generation of “Artificial Operons” and the Selection of Operons based on Size

The present invention contemplates a method for generating DNA sequences that would serve as artificial operons amenable to use with eukaryotic or prokaryotic genes. Some embodiments take advantage of the fact that multiple linked genes, each with its own individual promoter sequence, such as T7, and all within the same host or region, would be transcribed and translated to yield products identical to the products obtained from transcription and translation of an operon.

In some embodiments, single-stranded T7-linked cDNA (or other promoter-linked cDNA) that has been size selected to exclude unligated T7 sequences would generate artificial operons. The 5′ ends of the T7-cDNA are phosphorylated. Next, T4 RNA ligase is used to ligate the 5′ phosphate of the T7-cDNA to the free 3′ hydroxyl of another T7-cDNA, forming T7-cDNA concatamers. Generation of double-stranded cDNA would occur as described above by extending a primer complementary to the promoter. Concatamers could be size selected for the average length of a defined number of genes (to obtain “operons” with an average of two, three, four genes, etc.). These could be cloned as described above.

The preceding examples are included to demonstrate specific embodiments of the invention. It should be appreciated by those of skill in the art that the techniques disclosed in the examples which follow represent techniques discovered by the inventor to function well in the practice of the invention, and thus can be considered to constitute preferred modes for its practice. However, it should be understood that the invention is not intended to be limited to the particular forms disclosed. Rather, the different aspects of the disclosed compositions and methods may be utilized in various combinations and/or independently. Thus the invention is not limited to only those combinations shown herein, but rather may include other combinations. Further, those of skill in the art should, in light of the present disclosure, appreciate that many changes can be made in the specific embodiments which are disclosed and still obtain a like or similar result without departing from the spirit and scope of the invention.

### REFERENCES

The following references, to the extent that they provide exemplary procedural or other details supplementary to those set forth herein, are specifically incorporated herein by reference.

1. Caruso, F., Caruso, R. A., and Möhwald H. 1999 Chem. Mat. 11: 3309-3314.

2. Doi, N. and Yanagawa, H. 1999. FEBS Lett. 457(2): 227-230.

3. Kreider, B. L. 2000, Med Res Rev 20(3): 212-215.

4. Smith, G. P. 1985, Science 228:1315-1317.

5. Tawfik, D. S. and Griffiths, A. D. 1998. Nature Biotechnology 16: 652-656.

What is claimed:

1. A method for isolating DNA, RNA, and protein sequences, the method comprising the steps of:
formulating a solution comprising one or more components for in vitro transcription/translation reactions; and
encapsulating said solution such that transcription/translation reactions proceed inside said capsule.

2. The method of claim 1, wherein said capsule is a lipid vesicle.

3. The method of claim 2, wherein said lipid vesicle is adapted for identification based on a fluorescent activity.

4. The method of claim 1, wherein said capsule is a polymer capsule.

5. The method of claim 4, wherein said polymer capsule is adapted for identification based on a fluorescent activity.

6. The method of claim 1, wherein the step of encapsulating said solution such that transcription/translation reactions proceed inside said capsule further comprises encapsulating one or more microspheres inside said capsule, wherein said one or more microspheres are adapted to bond with one or more protein sequences.

7. The method of claim 1, wherein the step of encapsulating said solution such that transcription/translation reactions proceed inside said capsule further comprises encapsulating one or more microspheres inside said capsule, wherein said one or more microspheres are adapted to bond with one or more gene sequences.

8. The method of claim 7, wherein said one or more gene sequences comprise RNA.

9. The method of claim 7, wherein said one or more gene sequences comprise cDNA.

10. The method of claim 7, wherein said one or more gene sequences comprise DNA.

11. The method of claim 1, further comprising the step of:
isolating one or more transcription/translation reaction products from disrupted said one or more capsules.

12. The method of claim 1, wherein said one or more components are obtained from a eukaryote source.

13. The method of claim 1, wherein said one or more components are obtained from a prokaryote source.

14. A method for screening co-isolates, said method comprising the steps of:
formulating a solution comprising one or more components;
encapsulating said solution, said capsule containing said solution hereinafter referred to as a reactosome;
screening said reactosome for one or more specific activities; and
isolating said one or more reactosomes.

15. The method of claim 14, wherein said solution comprises a substrate for a metabolic pathway.

16. The method of claim 14, wherein said solution comprises a reporter for a metabolic pathway.

17. The method of claim 14, wherein said capsule comprises a lipid vesicle.

18. The method of claim 14, wherein said capsule comprises a polymer capsule.

19. The method of claim 14, wherein said step of isolating said one or more reactosomes comprises isolating said one or more reactosomes with metabolite product of enzymatic reactions.

20. The method of claim 19, further comprising the step of:
isolating one or more genes responsible for metabolite production.

21. The method of claim 14, wherein the step of screening said reactosome for one or more specific activities comprises screening said reactosome for fluorescent activity.

22. The method of claim 21, wherein screening said bound microspheres comprises screening for fluorescent activity.

23. The method of claim 6, wherein formulating said solution comprises adding one or more microspheres.

24. A method for selecting microspheres exhibiting a desired activity, said method comprising the steps of:
formulating a solution comprising one or more components, said one or more components comprising bound microspheres;
screening said bound microspheres; and
separating bound microspheres exhibiting one or more specific activities.

25. The method of claim 24, wherein said bound microspheres are manufactured by the method of claim 4.

26. The method of claim 24, wherein said bound microspheres are bound with a small molecule.

27. The method of claim 24, wherein screening said bound microspheres comprises screening for fluorescent activity.

28. The method of claim 24, wherein separating said bound microspheres comprises selecting said bound microspheres based on fluorescent activity.

29. A method for generating one or more gene sequences adapted for use as an operon, said method comprising the steps of:
obtaining a promoter-linked cDNA of selected size;
phosphorylating one or more ends of said promoter-linked cDNA;
ligating said promoter-linked cDNA such that a cDNA concatamer is formed; and
extending a selected primer complementary to said promoter-linked cDNA.

30. The method of claim 29, further comprising the generation of double-stranded cDNA.
